(19) United States
(12) Patent Application Publication
(10) Pub. No.: US 2025/0163189 A1
(43) Pub. Date: May 22, 2025

(54) RAFT AGENT FOR PHOTO-CONTROLLED POLYMERIZATION, PREPARATION METHOD THEREFOR, AND APPLICATIONS THEREOF

(71) Applicant: SHANDONG UNIVERSITY, Qingdao (CN)

(72) Inventors: Chenyu WU, Qingdao (CN); Wenjian LIU, Qingdao (CN); Zipeng ZHANG, Qingdao (CN)

(21) Appl. No.: 18/941,248

(22) Filed: Nov. 8, 2024

(30) Foreign Application Priority Data
Nov. 20, 2023 (CN) 202311553205.8

(51) Int. Cl. C08F 2/50; C07D 231/56

(52) U.S. Cl. CPC C08F 2/50; C07D 231/56; C08F 2438/03

(57) ABSTRACT

A RAFT agent for photo-controlled (iniferter) polymerization, preparation method therefor, and applications thereof. The RAFT agent has a structure represented by the general formula:

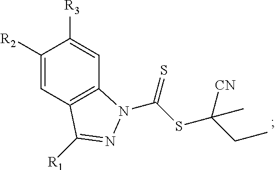

wherein R₂ and R₃ are each hydrogen, and R₁ is methyl. The RAFT agent is structurally and chemically stable, simple to synthesize, and enables rapid photo-controlled polymerization under blue light. It has been demonstrated that when using this RAFT agent for polymerization, the reaction time is reduced to just a few minutes, achieving a high conversion rate for acrylate and acrylamide monomers. Additionally, polymerization with the RAFT agent does not require the introduction of additional initiators or photocatalysts, as the RAFT agent itself can facilitate chain initiation and chain transfer under blue light. Compared to other photo-controlled polymerization methods, the apparent propagation rate is faster, and no additional impurities are introduced into the final product, thereby not affecting the color and performance of the polymer.

### CROSS-REFERENCE TO RELATED APPLICATIONS

The present invention claims priority to Chinese Patent Application No. 202311553205.8, titled “RAFT agent for photo-controlled polymerization, preparation method therefor, and applications thereof,” filed on Nov. 20, 2023, with the Chinese National Intellectual Property Administration, the entire content of which is incorporated herein by reference.

### TECHNICAL FIELD

The present invention relates to the technical field of RAFT agent synthesis, specifically to a RAFT agent for photo-controlled polymerization, preparation method therefor, and applications thereof.

### BACKGROUND OF THE INVENTION

The disclosure of the background art section is merely intended to enhance the overall understanding of the present invention and should not necessarily be considered as an acknowledgment or any form of suggestion that the information constitutes prior art that is already known to those skilled in the art.

RAFT polymerization is an abbreviation for Reversible Addition-Fragmentation Chain Transfer Polymerization, which allows precise control over the structure of polymers, including average molecular weight, polydispersity, and specific functionalities. This control enables the synthesis of polymers with customized properties and specific structures, such as block copolymers, star polymers, and gradient copolymers. RAFT polymerization is compatible with various monomers, including hydrophilic monomers, hydrophobic monomers, and various functional monomers. Moreover, RAFT polymerization can be carried out in a wide range of solvents, including both polar and non-polar solvents. Overall, the advantages of RAFT polymerization make it a powerful tool for synthesizing polymers with specific functions and chain lengths, enhancing control over macromolecular structure and function, and it is widely used in academic research and industrial production.

The combination of photo-controlled polymerization and RAFT polymerization allows temporal and spatial control over the polymerization process, thereby facilitating the attainment of target molecular weights and low molecular weight dispersity polymers. Photo-controlled RAFT polymerization is a technique that utilizes light to initiate, regulate, and terminate the polymerization process. Compared to traditional methods, it offers spatio-temporal control, low energy consumption, fewer by-products, etc. In recent years, the field of photo-controlled polymerization has developed rapidly and has been widely applied in material science, biomedicine, nanotechnology, etc. However, current challenges in photo-controlled RAFT polymerization are mainly on sluggish apparent propagation rates and additional requirement of catalytic species such as photocatalysts.

### SUMMARY OF THE INVENTION

To overcome the deficiencies in the prior art, the present invention aims to provide a RAFT agent for photo-controlled (iniferter) polymerization, preparation method therefor, and applications thereof. The RAFT agent provided by the present invention is structurally and chemically stable, simple to synthesize, and capable of rapid photo-controlled polymerization.

To achieve the above objectives, the technical solution of the present invention is as follows.

In a first aspect, the present invention provides a RAFT agent for photo-controlled polymerization, wherein a structure of the RAFT agent for photo-controlled polymerization is represented by the general formula:

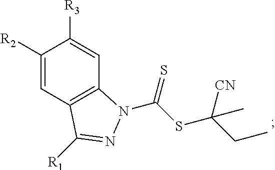

wherein, R₁, R₂, and R₃ are each hydrogen;
or R₂ and R₃ are each hydrogen and R₁ is methyl;
or R₁ is methyl, R₂ is bromine, and R₃ is hydrogen;
or R₁ is methyl, R₂ is hydrogen, and R₃ is methoxy.

In some embodiments of the present invention, R₂ and R₃ are each hydrogen, and R₁ is methyl.

In a second aspect, the present invention provides a method for preparing the aforementioned RAFT agent for photo-controlled polymerization, comprising:
dissolving

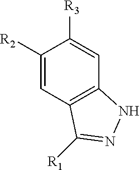

and a metal hydroxide in a first solvent to form a reaction mixture, and keeping the reaction mixture in an ice water bath, then adding carbon disulfide dropwise to the reaction mixture and continuously stirring the reaction mixture in the ice water bath to obtain an intermediate product I;
dissolving the intermediate product I in a second solvent, adding an oxidizing agent, and stirring to obtain an intermediate product II;
dissolving the intermediate product II and 2,2′-Azobisisovaleronitrile (AIBN) in a third solvent, heating, stirring, and introducing nitrogen gas; after the reaction is complete, obtaining the RAFT agent for photo-controlled polymerization;
wherein a structure of the intermediate product I is shown as formula I, and a structure of the intermediate product II is shown as formula II;

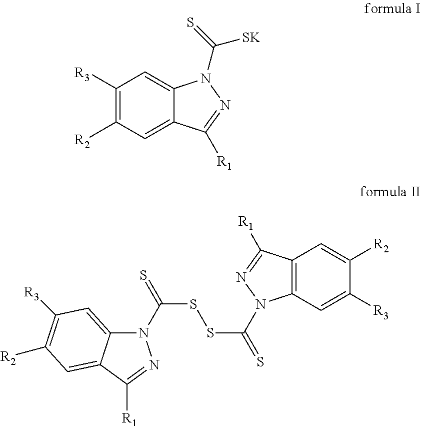

wherein, R₁, R₂, and R₃ are each hydrogen;
or R₂ and R₃ are each hydrogen, and R₁ is methyl;
or R₁ is methyl, R₂ is bromine, and R₃ is hydrogen;
or R₁ is methyl, R₂ is hydrogen, and R₃ is methoxy.

In some embodiments of the present invention, the metal hydroxide is selected from at least one of potassium hydroxide and sodium hydroxide.

In some embodiments of the present invention, the first solvent, the second solvent and the third solvent are respectively selected from tetrahydrofuran, ethyl acetate and acetone.

In some embodiments of the present invention, the first solvent and the second solvent are tetrahydrofuran, and the third solvent is ethyl acetate.

In some embodiments of the present invention, after adding carbon disulfide to the reaction mixture and stirring the reaction mixture, a product is obtained, filtered under reduced pressure, washed with deionized water, and dried to yield the intermediate product I.

In some embodiments of the present invention, after adding the oxidizing agent and stirring to complete the reaction, the second solvent and the oxidizing agent are removed, followed by extraction and drying to yield the intermediate product II.

In some embodiments of the present invention, the oxidizing agent comprises elemental iodine.

In some embodiments of the present invention, a rotary evaporation method is used to remove the second solvent.

In some embodiments of the present invention, a reducing agent is added to remove the oxidizing agent; preferably, the reducing agent comprises sodium thiosulfate.

In some embodiments of the present invention, after heating, stirring, and introducing nitrogen gas until the reaction is complete, the third solvent is removed, and a product is obtained, followed by purification via column chromatography to yield the RAFT agent for photo-controlled polymerization.

Preferably, during the purification via column chromatography, a mixture of petroleum ether and ethyl acetate in a volume ratio of 10:1 is used as a mobile phase.

In a third aspect, the present invention provides an application of the aforementioned RAFT agent in a RAFT polymerization reaction, wherein the RAFT polymerization reaction is a photo-controlled polymerization reaction.

In some embodiments of the present invention, the photo-controlled polymerization reaction comprises mixing a monomer, a RAFT agent for photo-controlled polymerization described in the first aspect, and a solvent uniformly, followed by a light-induced reaction under 420-430 nm blue light to obtain a polymer.

In some embodiments of the present invention, the solvent includes, but is not limited to, acetonitrile, methanol, N,N-dimethylformamide, tetrahydrofuran, ethyl acetate, ethanol, dichloromethane, acetone, dimethyl sulfoxide.

In a fourth aspect, the present invention provides a method for photo-controlled RAFT polymerization, comprising:
mixing a monomer, a RAFT agent, and a solvent;
performing a photo-controlled RAFT polymerization reaction under blue light irradiation at 420-430 nm; and
isolating and purifying the resulting polymer.
wherein, the RAFT agent is as described in the first aspect.

In some embodiments of the present invention, the monomer is selected from acrylate monomers or acrylamide monomers.

In some embodiments of the present invention, the solvent includes, but is not limited to, acetonitrile, methanol, N,N-dimethylformamide, tetrahydrofuran, ethyl acetate, ethanol, dichloromethane, acetone, dimethyl sulfoxide.

### Beneficial Effects of the Invention

The RAFT agent provided by the present invention is structurally and chemically stable, simple to synthesize, and capable of rapid photo-controlled polymerization under blue light. It has been verified that when using the RAFT agent provided by the present invention for polymerization, the reaction time is only a few minutes, and acrylate and acrylamide monomers can achieve high conversion rates, which is much shorter than the reaction times of other photo-controlled polymerization methods (such as photoinduced electron/energy transfer RAFT polymerization and photo-controlled atom transfer radical polymerization). Moreover, when using the RAFT agent provided by the present invention for polymerization, there is no need to introduce additional initiators or photocatalysts; the RAFT agent itself can initiate and transfer chains under blue light. Compared to other photo-controlled polymerization methods (such as photoinduced electron/energy transfer RAFT polymerization and photo-controlled atom transfer radical polymerization), the apparent propagation rate is faster, and no additional impurities are introduced into the final product on account of its catalyst-free feature, thereby not affecting the color or performance of the polymer.

### BRIEF DESCRIPTION OF DRAWINGS

The accompanying drawings, which constitute a part of the present invention, are provided to further understand the present invention. The exemplary embodiments of the present invention and their descriptions are used to explain the present invention and do not constitute undue limitations on the present invention.

FIG. 1 is a ¹H-NMR spectrum of RAFT1 (Z1) prepared in example 1 of the present invention.

FIG. 2 is a ¹H-NMR spectrum of RAFT2 (Z2) prepared in example 2 of the present invention.

FIG. 3 is a ¹H-NMR spectrum of RAFT3 (Z3) prepared in example 3 of the present invention.

FIG. 4 is a ¹H-NMR spectrum of RAFT4 (Z4) prepared in example 4 of the present invention.

FIG. 5 is UV-Vis absorption spectra of RAFT agents prepared in the examples of the present invention, which can be used as a basis for selecting the light source.

FIG. 6 shows a ¹H-NMR spectrum for end-group verification of RAFT2 (Z2).

FIG. 7 is kinetic curves of RAFT2 (Z2) polymerization at different wavelengths.

FIG. 8A is a kinetic curve of RAFT2 (Z2) under anaerobic conditions.

FIG. 8B is a graph of Mn and Mw/Mn changes with conversion rate under anaerobic conditions for RAFT2 (Z2).

FIG. 8C is a GPC molecular weight distribution graph of RAFT2 (Z2) under anaerobic conditions.

FIG. 8D is a kinetic curve of RAFT2 (Z2) under oxygen conditions.

FIG. 8E is a graph of Mn and Mw/Mn changes with conversion rate under oxygen conditions for RAFT2 (Z2).

FIG. 8F is a GPC molecular weight distribution graph of RAFT2 (Z2) under oxygen conditions.

FIG. 9 is kinetic curves of RAFT2 (Z2) polymerization with different ratios.

FIG. 10 is GPC graphs of RAFT2 (Z2) with different ratios.

### DETAILED DESCRIPTION OF THE INVENTION

Embodiments of the present invention provide a RAFT agent, wherein Z group of the RAFT agent is an indazole-based compound group, and R group is a structure that has been previously reported. A general structural formula of the RAFT agent for photo-controlled polymerization is as follows:

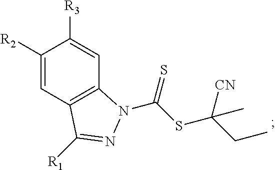

wherein, R₁, R₂, and R₃ are each hydrogen;
or R₂ and R₃ are each hydrogen, and R₁ is methyl;
or R₁ is methyl, R₂ is bromine, and R₃ is hydrogen;
or R₁ is methyl, R₂ is hydrogen, and R₃ is methoxy.

In this embodiment, when R₁, R₂, and R₃ are each hydrogen, the Z group is an indazole group. To distinguish the Z group from other types of Z groups, it is named Z1 group, and a structure of the Z1 group is as follows:

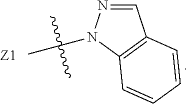 [SMILES: *n1ncc2ccccc21]

In this embodiment, when R₂ and R₃ are each hydrogen and R₁ is methyl, the Z group is a methyl-substituted indazole group. The Z group is named Z2 group, and a structure of the Z2 group is as follows:

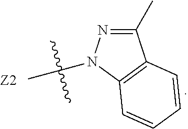 [SMILES: *n1nc(C)c2ccccc21]

In this embodiment, when R₁ is methyl, R₂ is bromine, and R₃ is hydrogen, the Z group is a doubly substituted indazole group. The Z group is named Z3 group, and a structure of the Z3 group is as follows:

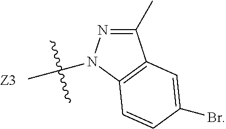 [SMILES: *n1nc(C)c2cc(Br)ccc21]

In this embodiment, when R₁ is methyl, R₂ is hydrogen, and R₃ is methoxy, the Z group is a doubly substituted indazole group. The Z group is named Z4 group, and a structure of the Z4 group is as follows:

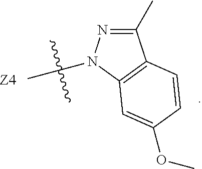 [SMILES: *n1nc(C)c2ccc(OC)cc21]

Embodiments of the present invention also provides a method for preparing the RAFT agent described in the present invention, comprising:
dissolving

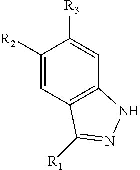

and a metal hydroxide in a first solvent to form a reaction mixture, and keeping the reaction mixture in an ice water bath, then adding carbon disulfide dropwise to the reaction mixture and continuously stirring the reaction mixture in the ice water bath to obtain an intermediate product I;
dissolving the intermediate product I in a second solvent, adding an oxidizing agent, and stirring to obtain an intermediate product II;
dissolving the intermediate product II and 2,2′-Azobisisovaleronitrile (AIBN) in a third solvent, heating, stirring, and introducing nitrogen gas; after the reaction is complete, obtaining the RAFT agent for photo-controlled polymerization;
wherein a structure of the intermediate product I is shown as formula I, and a structure of the intermediate product II is shown as formula II;

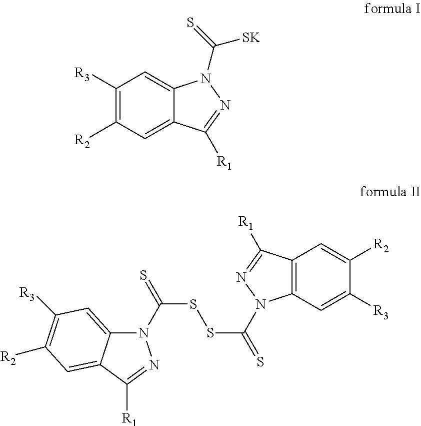

wherein, R₁, R₂, and R₃ are each hydrogen;
or R₂ and R₃ are each hydrogen, and R₁ is methyl;
or R₁ is methyl, R₂ is bromine, and R₃ is hydrogen;
or R₁ is methyl, R₂ is hydrogen, and R₃ is methoxy.

In this embodiment, the options for the

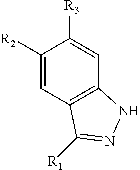

group include indazole, 3-methyl-1H-indazole, 5-bromo-3-methyl-1H-indazole, or 6-methoxy-3-methyl-1H-indazole.

The method provided by the embodiments of the present invention is simple and efficient, and the resulting RAFT agent is structurally and chemically stable, capable of rapid photo-controlled polymerization, suitable for various monomers, and does not introduce additional groups into the final product, thereby not affecting the properties of the final product.

In this embodiment, a suitable solvent including the first solvent, the second solvent and the third solvent, such as tetrahydrofuran, ethyl acetate or acetone, can be selected according to actual needs.

In one embodiment, the metal hydroxide is selected from at least one of potassium hydroxide and sodium hydroxide, but is not limited thereto.

In one embodiment, after adding carbon disulfide to the reaction mixture and stirring the reaction mixture, a product is obtained, filtered under reduced pressure, washed with deionized water, and dried to yield the intermediate product I.

In one embodiment, after adding the oxidizing agent and stirring to complete the reaction, the second solvent and the oxidizing agent are removed, followed by extraction and drying to yield the intermediate product II.

Wherein the oxidizing agent includes, but is not limited to, oxygen, chlorine, elemental bromine, elemental iodine, and the like.

Wherein, a rotary evaporation method is used to remove the second solvent.

Wherein, a reducing agent can be added to remove the oxidizing agent.

Wherein, the reducing agent includes but is not limited to sodium thiosulfate, ascorbic acid (vitamin C), ammonium iron (II) sulfate, etc.

In one embodiment, after heating, stirring, and introducing nitrogen gas until the reaction is complete, the third solvent is removed, and a product is obtained, followed by purification via column chromatography to yield the RAFT agent for photo-controlled polymerization.

During the purification via column chromatography, a mixture of petroleum ether and ethyl acetate in a volume ratio of 10:1 is used as a mobile phase.

Wherein a temperature of heating is 70-80° C.

Embodiments of the present invention also provide an application of the RAFT agent described herein in a RAFT polymerization reaction, wherein the RAFT polymerization reaction is a photo-controlled polymerization reaction. In this embodiment, using the RAFT agent for the RAFT polymerization reaction is applicable to the polymerization of various monomers without introducing additional groups into the final product, thus not adversely affecting the properties of the final product. Most importantly, when using the RAFT agent of the present invention in a RAFT polymerization reaction, the apparent propagation rate of the monomer can be significantly increased. For the model monomer methyl acrylate, a monomer conversion rate of over 77% can be achieved within 3 minutes of blue light irradiation.

In a further embodiment, the photo-controlled polymerization reaction comprises mixing a monomer, the RAFT agent described above, and a solvent uniformly, followed by a light-induced reaction under 420-430 nm blue light to obtain a polymer. When using the RAFT agent provided by the present invention for photo-controlled polymerization, there is no need to add additional photo initiators, and no additional groups are introduced into the final product, thereby not affecting the properties of the final product.

To enable those skilled in the art to more clearly understand the technical solutions of the present invention, the following specific embodiments are provided to further illustrate the technical solutions of the present invention.

### Example 1

### Synthesis of RAFT1 (Z1)

The synthesis route was as follows:

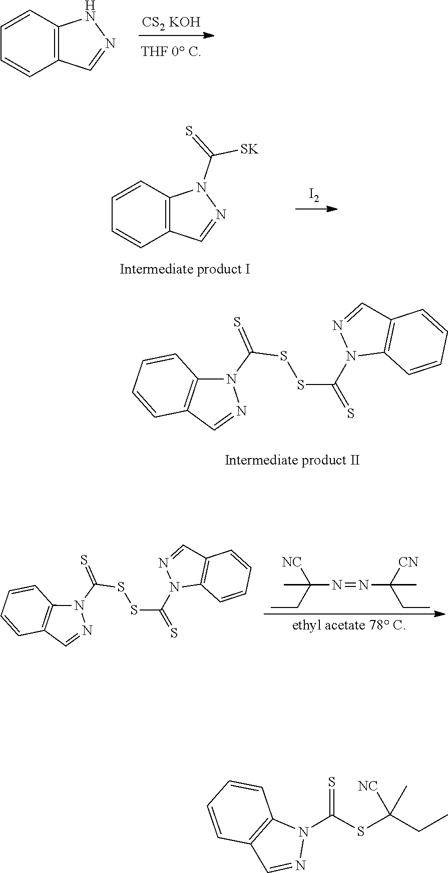

(1) Indazole (2 g) and potassium hydroxide (960 mg) were dissolved in 200 mL of tetrahydrofuran, and the reaction system was kept in an ice water bath for 15 minutes. Then, carbon disulfide (0.73 mL) was added dropwise while stirring continuously in the ice water bath for 4-6 hours. The product was filtered under reduced pressure, washed with deionized water 3-5 times, and dried to obtain intermediate product I.

(2) Intermediate product I (2 g) was dissolved in tetrahydrofuran, then elemental iodine (2.2 g) was added and stirred continuously for 12 hours. The product was then evaporated to remove tetrahydrofuran, and an appropriate amount of sodium thiosulfate aqueous solution was added to remove excess elemental iodine. The product was extracted with ether, dried, and obtained as intermediate product II.

(3) Intermediate product II (2 g) and 2,2′-Azobisisovaleronitrile (AIBN) (1.85 g) were dissolved in 200 mL of ethyl acetate, and the mixture was heated and stirred continuously at 78° C. for 16 hours while continuously introducing nitrogen gas. After heating was completed, the product was rotary evaporated to remove the ethyl acetate, and the product was purified by column chromatography. The mobile phase was a mixture of petroleum ether and ethyl acetate in a volume ratio of 10:1.

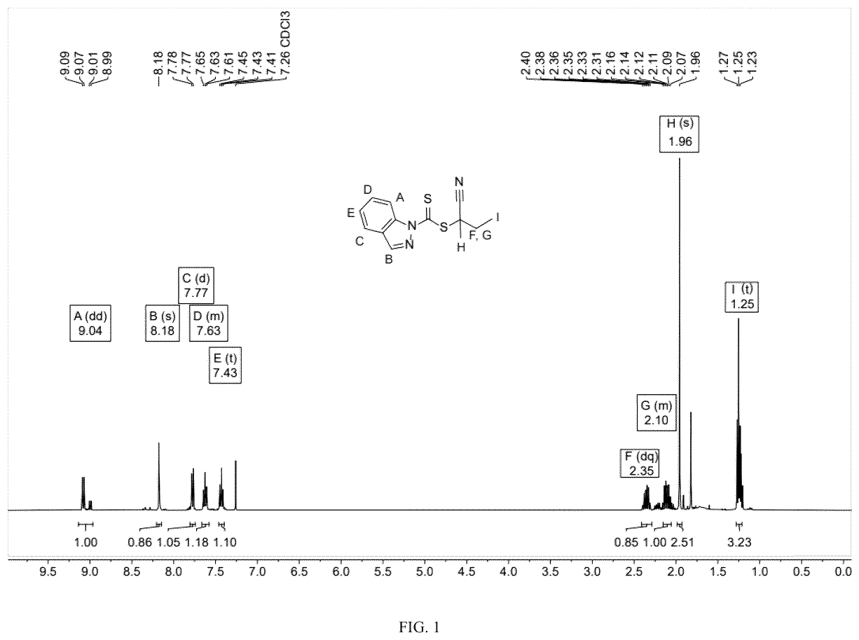

As shown in FIG. 1, RAFT1 (Z1) was successfully synthesized as verified.

### Example 2: Synthesis of RAFT2 (Z2)

The synthesis route for RAFT2 (Z2) was the same as for RAFT1 (Z1), except for the initial materials.

(1) 3-Methyl-1H-indazole (2 g) and potassium hydroxide (850 mg) were dissolved in 200 mL of tetrahydrofuran, and the reaction system was kept in an ice water bath for 20 minutes. Then, carbon disulfide (0.87 mL) was added dropwise while stirring continuously in the ice water bath for 10-12 hours. The product was filtered under reduced pressure, washed with deionized water 3-5 times, and dried to obtain intermediate product I.

(2) Intermediate product 1 (2 g) was dissolved in tetrahydrofuran, then elemental iodine (2.06 g) was added and stirred continuously for 24 hours. The product was then evaporated to remove tetrahydrofuran, and an appropriate amount of sodium thiosulfate aqueous solution was added to remove excess elemental iodine. The product was extracted with ether, dried, and obtained as intermediate product II.

(3) Intermediate product 11 (2 g) and 2,2′-Azobisisovaleronitrile (AIBN) (1.85 g) were dissolved in 200 mL of ethyl acetate, heated and stirred continuously at 78° C. for 24 hours, with nitrogen gas continuously introduced. After heating, the product was evaporated to remove ethyl acetate and purified by column chromatography. The mobile phase was a mixture of petroleum ether and ethyl acetate in a volume ratio of 12:1.

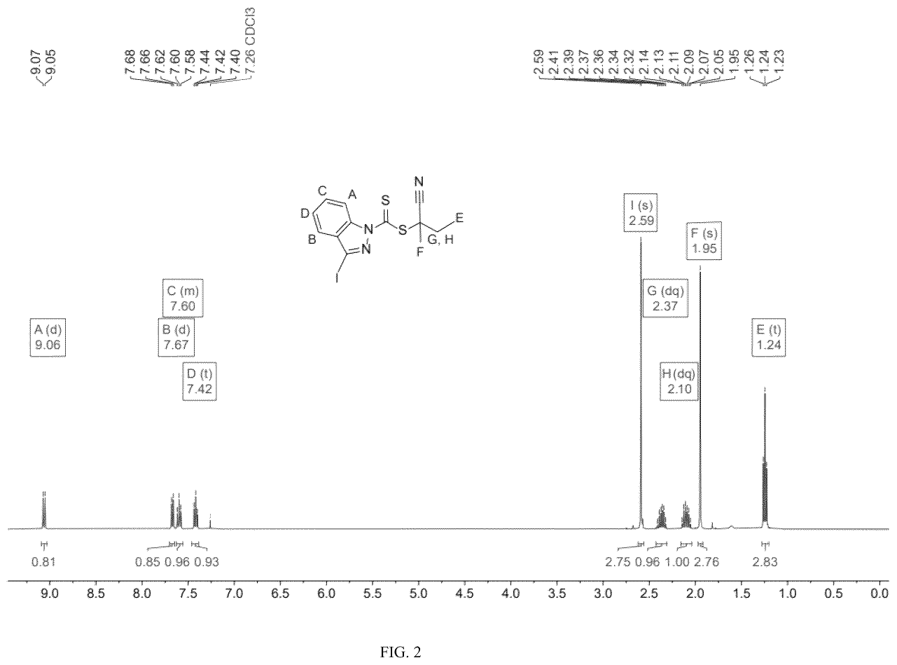

As shown in FIG. 2, RAFT2 (Z2) was successfully synthesized as verified.

### Example 3: Synthesis of RAFT3 (Z3)

The synthesis route for RAFT3 (Z3) was the same as for RAFT1 (Z1), except for the initial materials.

(1) 5-Bromo-3-methyl-1H-indazole (2 g) and potassium hydroxide (532 mg) were dissolved in 200 mL of tetrahydrofuran, and the reaction system was kept in an ice water bath for 15 minutes. Then, carbon disulfide (0.57 mL) was added dropwise while stirring continuously in the ice water bath for 6-8 hours. The product was filtered under reduced pressure, washed with deionized water 3-5 times, and dried to obtain intermediate product I.

(2) Intermediate product 1 (2 g) was dissolved in tetrahydrofuran, then elemental iodine (1.56 g) was added and stirred continuously for 12 hours. The product was then evaporated to remove tetrahydrofuran, and an appropriate amount of sodium thiosulfate aqueous solution was added to remove excess elemental iodine. The product was extracted with ether, dried, and obtained as intermediate product II.

(3) Intermediate product II (2 g) and 2,2′-Azobisisovaleronitrile (AIBN) (0.672 g) were dissolved in 200 mL of ethyl acetate, heated and stirred continuously at 78° C. for 24 hours, with nitrogen gas continuously introduced. After heating, the product was evaporated to remove ethyl acetate and purified by column chromatography. The mobile phase was a mixture of petroleum ether and ethyl acetate in a volume ratio of 10:1.

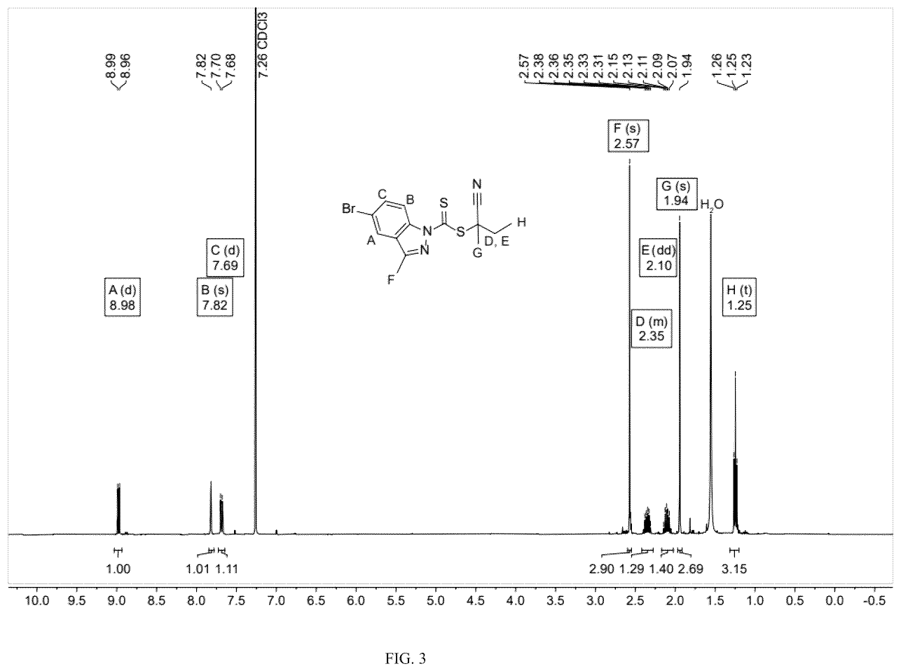

As shown in FIG. 3, RAFT3 (Z3) was successfully synthesized verified.

### Example 4: Synthesis of RAFT4 (Z4)

The synthesis route for RAFT4 (Z4) was the same as for RAFT1 (Z1), except for the initial materials.

(1) 6-Methoxy-3-methyl-1H-indazole (2 g) and potassium hydroxide (692 mg) were dissolved in 200 mL of tetrahydrofuran, and the reaction system was kept in an ice water bath for 15 minutes. Then, carbon disulfide (0.742 mL) was added dropwise while stirring continuously in the ice water bath for 6-8 hours. The product was filtered under reduced pressure, washed with deionized water 3-5 times, and dried to obtain intermediate product I.

(2) Intermediate product I (2 g) was dissolved in tetrahydrofuran, then elemental iodine (1.84 g) was added and stirred continuously for 12 hours. The product was then evaporated to remove tetrahydrofuran, and an appropriate amount of sodium thiosulfate aqueous solution was added to remove excess elemental iodine. The product was extracted with ether, dried, and obtained as intermediate product II.

(3) Intermediate product II (2 g) and 2,2′-Azobisisovaleronitrile (AIBN) (810 mg) were dissolved in 200 mL of ethyl acetate, heated and stirred continuously at 78° C. for 16 hours, with nitrogen gas continuously introduced. After heating, the product was evaporated to remove ethyl acetate and purified by column chromatography. The mobile phase was a mixture of petroleum ether and ethyl acetate in a volume ratio of 15:1.

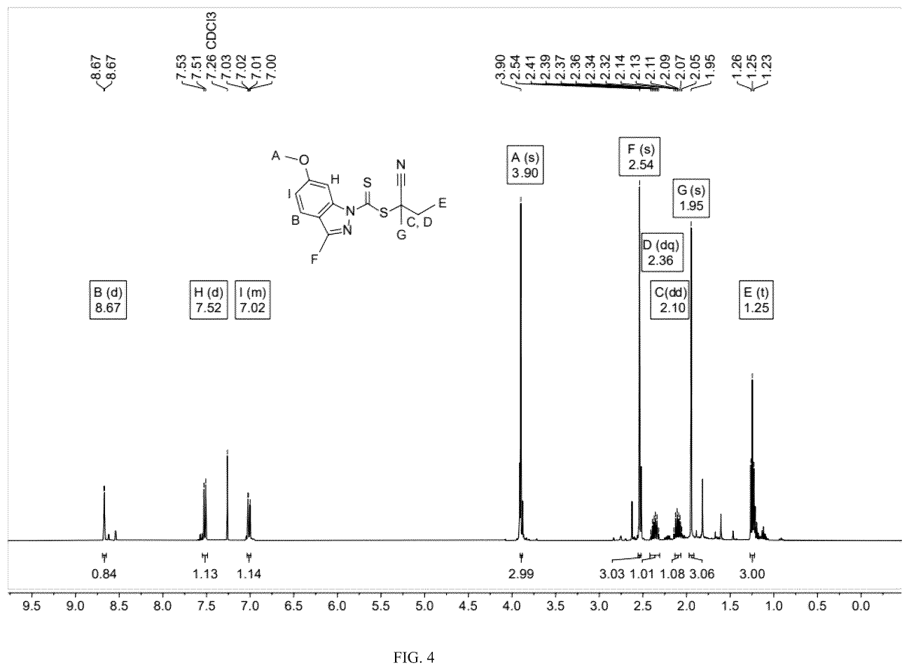

As shown in FIG. 4, RAFT4 (Z4) was successfully synthesized as verified.

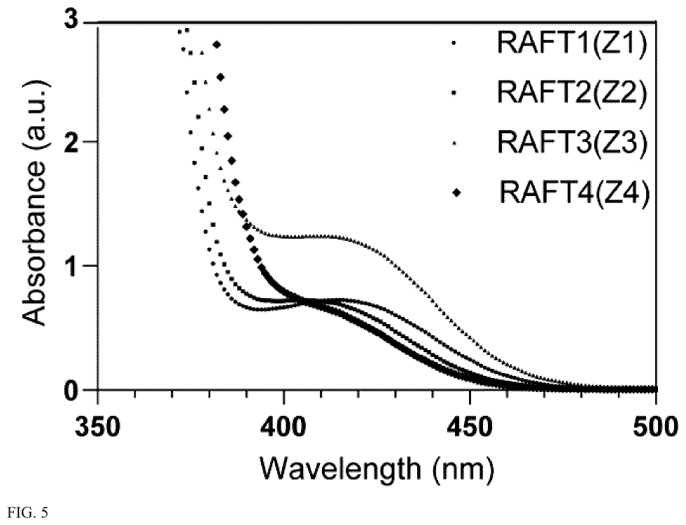

FIG. 5 shows the UV-Vis absorption spectra of the RAFT agents prepared in Examples 1-4, which can be used as a basis for selecting the light source.

### Example 5: Verification of Apparent Propagation Rates for RAFT Agents Prepared in Examples 1-4

### 1. Kinetic Experiments of Photo-Controlled RAFT Polymerization Under Blue Light

1.1 Preparation of Kinetic Solution: A solution was prepared with a molar ratio of [monomer]:[RAFT agent]=200:1 by mixing the monomer and the RAFT agent, with dimethyl sulfoxide (DMSO) as the solvent. The volume ratio of monomer to solvent was 1:1. The monomer used was methyl acrylate (MA), and the RAFT agents were those prepared in examples 1-4, respectively.

1.2 Kinetic Experiment Procedure: 0.4 mL of the kinetic solution was transferred into a cuvette, sealed with a rubber stopper and sealing film. The solution was irradiated under a 420-430 nm, 10 mW/cm² LED light. Fourier Transform Infrared Spectroscopy (FTIR) was used to integrate the vibration absorption peak of the vinyl C—H bond of the monomer near 6200 cm⁻¹. The peak area was used for timed quantitative monitoring of the remaining monomer, thereby tracking the monomer conversion rate. The apparent propagation rate (kₚ^{app}) was calculated using a kinetic equation. Samples were taken when the monomer conversion rates were approximately 40%, 50%, 60%, and 70%, respectively. These samples with different conversion rates were analyzed by Gel Permeation Chromatography (GPC) to check whether the molecular weight during the polymerization process was controllable and to monitor the molecular weight dispersity of the polymer. The test results are shown in Table 1.

TABLE 1
Polymerization Performances of Different RAFT Agents

| | | Induction | | | | | |
|---|---|---|---|---|---|---|---|
| | kₚ^{app} | Period | | M_{n, Theo} | M_{n, GPC} | | Time |
| RAFT | (min⁻¹) | (min) | Conversion | (g/mol) | (g/mol) | Ð | (min) |
| RAFT1 | 0.503 | 0.85 | 65.1% | 11500 | 13200 | 1.12 | 3 |
| RAFT2 | 0.704 | 0.94 | 77.7% | 13700 | 13200 | 1.11 | 3 |
| RAFT3 | 0.567 | 0.81 | 71.2% | 13300 | 14500 | 1.11 | 3 |
| RAFT4 | 0.742 | 0.89 | 79.1% | 13900 | 15100 | 1.14 | 3 |

As shown in Table 1, the apparent propagation rates were ranked as follows: RAFT4>RAFT2>RAFT3>RAFT1. Based on the apparent propagation rates of the RAFT agents, and considering the higher synthesis cost of RAFT4, the RAFT agent used in the following examples is RAFT2 synthesized in Example 2.

### Example 6: Performance Verification of RAFT Agent Prepared in the Example

1. Kinetic Experiment of Photo-Controlled RAFT Polymerization Under Blue Light

1.1 Preparation of Kinetic Solution: A solution was prepared with a molar ratio of [monomer]:[RAFT agent]=200:1 by mixing the monomer and the RAFT agent, with dimethyl sulfoxide (DMSO) as the solvent. The volume ratio of monomer to solvent was 1:1. The monomer used was methyl acrylate (MA), and the RAFT agent was RAFT2 prepared in example 2.

1.2 Kinetic Experiment Procedure: 0.4 mL of the kinetic solution was transferred into a cuvette, sealed with a rubber stopper and sealing film. The solution was irradiated under a 10 mW/cm² LED light. Fourier Transform Infrared Spectroscopy (FTIR) was used to integrate the vibration absorption peak of the vinyl C—H bond of the monomer near 6200 cm⁻¹. The peak area was used for timed quantitative monitoring of the remaining monomer, thereby tracking the monomer conversion rate. The apparent propagation rate (kₚ^{app}) was calculated using a kinetic equation. Samples were taken when the monomer conversion rates were approximately 40%, 50%, 60%, and 70%, respectively. These samples with different conversion rates were analyzed by Gel Permeation Chromatography (GPC) to check whether the molecular weight during the polymerization process was controllable and to monitor the dispersity of the polymer. After the monomer conversion rate reaches approximately 70% (after the fourth sampling), the polymer was purified and then analyzed using Nuclear Magnetic Resonance (NMR) spectroscopy to verify the retention of the polymer chain end groups.

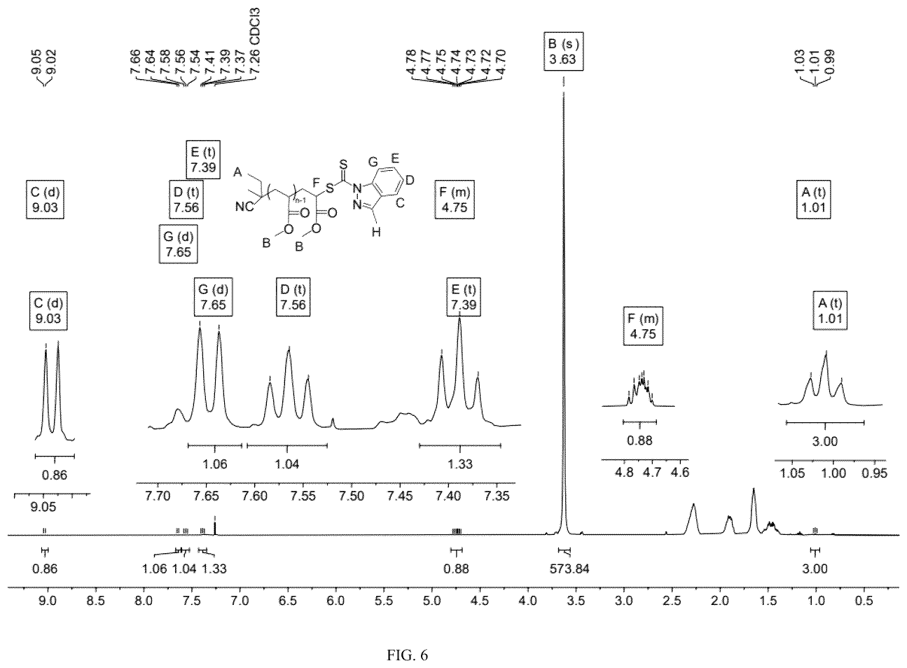

The results of the Nuclear Magnetic Resonance (NMR) spectroscopy are shown in FIG. 6, indicating that the polymer chain end groups retain the Z and R terminal groups of RAFT2 (Z2).

### 2. Kinetic Data of RAFT Agent (RAFT2 Prepared in Example 2) Under Different Wavelength LED Light in an Anaerobic Environment

The utilization of light energy as a renewable and clean energy source has always been a hot topic of research. To better harness light energy, we conducted ultraviolet-visible spectroscopy absorption measurements on different RAFT agent. We found that RAFT1-4 agents all exhibited absorption peaks in the 400-460 nm wavelength range. To utilize light energy more efficiently and fully, we screened for the absorption wavelength of the RAFT agent with the highest apparent propagation rate by conducting kinetic experiments at different wavelengths. Using the RAFT2 agent and methyl acrylate monomer as an example, we conducted kinetic experiments under different wavelengths. The specific experimental data are shown in Table 2.

TABLE 2
Polymerization Performances of RAFT Agent at Different Wavelengths

| | | Induction | | | | | |
|---|---|---|---|---|---|---|---|
| Wavelength | kₚ^{app} | Period | | M_{n, Theo} | M_{n, GPC} | | Time |
| (nm) | (min⁻¹) | (min) | Conversion | (g/mol) | (g/mol) | Ð | (min) |
| 400-410 | 0.446 | 1.02 | 71.6% | 12600 | 12300 | 1.11 | 3.5 |
| 410-420 | 0.677 | 0.98 | 74.8% | 13200 | 13100 | 1.14 | 3 |
| 420-430 | 0.690 | 0.92 | 75.6% | 13300 | 13800 | 1.15 | 3 |
| 430-440 | 0.665 | 0.95 | 75.6% | 13300 | 13800 | 1.14 | 3 |
| 440-450 | 0.403 | 1.65 | 74.1% | 13000 | 13300 | 1.09 | 5 |
| 450-460 | 0.217 | 2.73 | 68.8% | 12100 | 12200 | 1.07 | 8 |

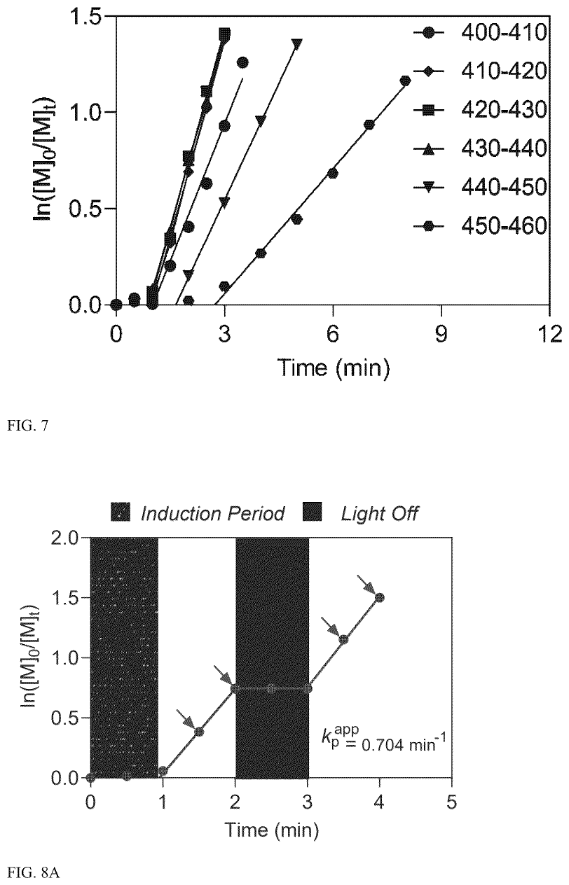

As shown in Table 2 and FIG. 7, the apparent propagation rate of the RAFT agent was highest at a wavelength of 420-430 nm. Therefore, subsequent experiments on aerobic and anaerobic environments, different ratios of RAFT agent, different solvents, and different monomers were conducted at a wavelength of 420-430 nm.

### 3. Comparison of RAFT Agent (RAFT2 Prepared in Example 2) in Aerobic and Anaerobic Environments Under 420-430 nm LED Light

In practical applications, the polymerization process is usually conducted in an open environment, making it difficult to achieve an anaerobic environment, which limits the application of RAFT agents. To better apply RAFT agents in various fields, it is necessary to address the issue of oxygen tolerance in photo-controlled RAFT polymerization. Since the polymerization mechanism itself belongs to free radical polymerization, it is easily affected by oxygen. Therefore, we conducted kinetic experiments on RAFT agents in aerobic and anaerobic environments to study their oxygen tolerance.

TABLE 3
The Effect of Oxygen on Polymerization of RAFT Agent Under 420-430 nm LED Light

| | | Induction | | | | | |
|---|---|---|---|---|---|---|---|
| | kₚ^{app} | Period | | M_{n, Theo} | M_{n, GPC} | | Time |
| | (min⁻¹) | (min) | Conversion | (g/mol) | (g/mol) | Ð | (min) |
| Anaerobic | 0.704 ± 0.0247 | 0.94 ± 0.02 | 77.7% | 13700 | 13200 | 1.11 | 3 |
| Aerobic | 0.575 ± 0.0068 | 0.60 ± 0.26 | 71.0% | 12500 | 14800 | 1.13 | 3 |

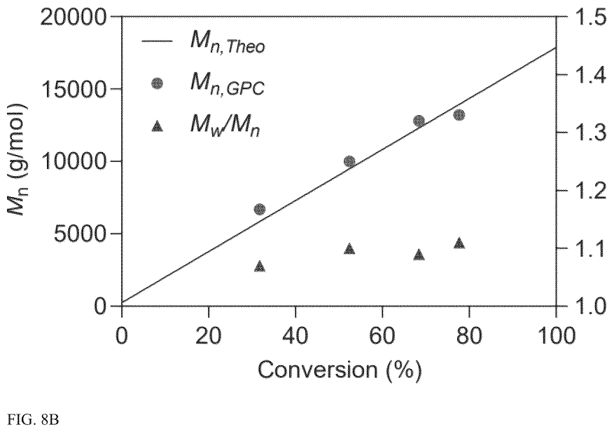

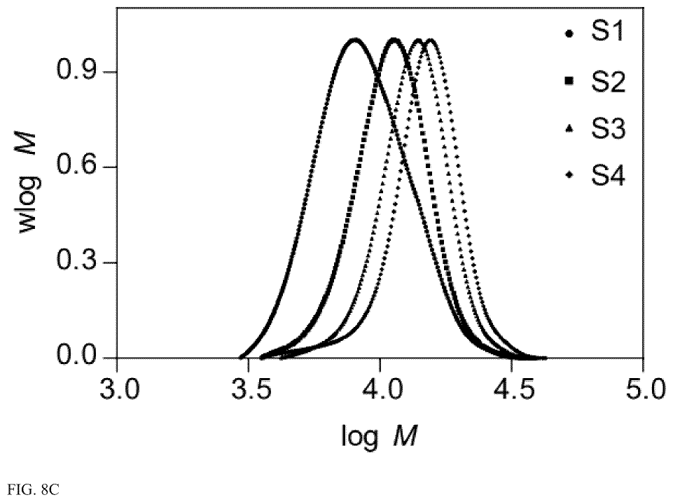

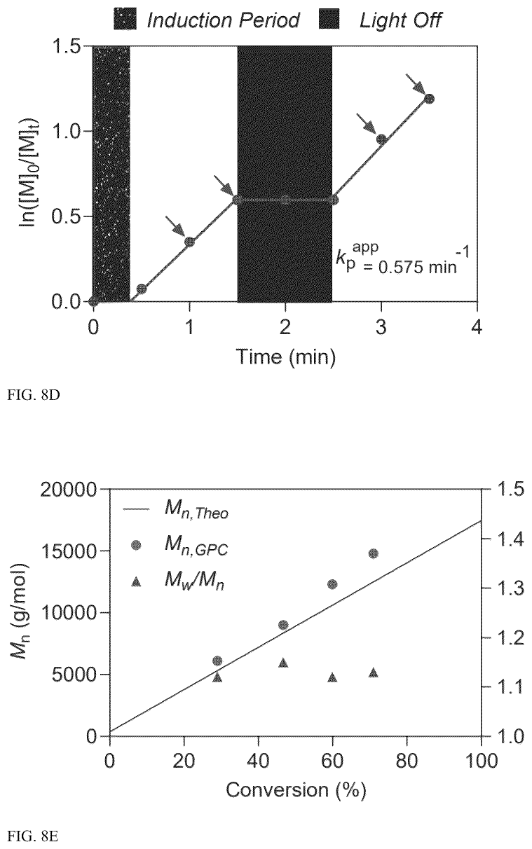

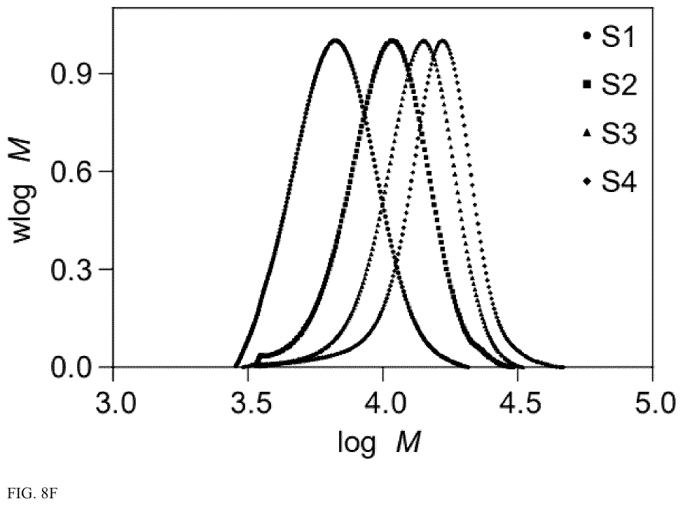

As shown in Table 3 and FIGS. 8A to 8I, the apparent propagation rate of monomers under aerobic conditions was lower than that under anaerobic conditions. RAFT polymerization follows the mechanism of free radical polymerization, where radicals generated by light are easily quenched by oxygen, resulting in a higher apparent propagation rate in anaerobic environments.

As shown in FIG. 8A and FIG. 8D, when the light was stopped, polymerization immediately stopped, and when the light was continued, polymerization continued, demonstrating excellent control over the polymerization process by light.

As shown in FIG. 8B and FIG. 8E, even with the participation of oxygen, the polymerization process remained controllable, with D values less than 1.2, indicating good control during polymerization even with oxygen present.

### 4. Polymerization of RAFT Agent (RAFT2 Prepared in Example 2) at Different Ratios in Anaerobic Environment

In previous experiments, the ratio of monomer to RAFT agent was consistently maintained at 200:1. For higher monomer ratios (such as 500:1 or 1000:1) or lower monomer ratios (such as 100:1), we conducted kinetic experiments to explore the controllable range of the RAFT agent. These experiments were carried out with different ratios of RAFT agent to monomer, studying the effects of various monomer and RAFT agent ratios.

TABLE 4
Kinetic Data of RAFT Agent at Different Ratios in Anaerobic Environment

| | | Induction | | | | | |
|---|---|---|---|---|---|---|---|
| | kₚ^{app} | Period | | M_{n, Theo} | M_{n, GPC} | | Time |
| Ratio | (min⁻¹) | (min) | Conversion | (g/mol) | (g/mol) | Ð | (min) |
| 100:1 | 0.802 | 1.35 | 73.1% | 6600 | 8700 | 1.11 | 3 |
| 200:1 | 0.704 | 0.94 | 77.7% | 13700 | 13200 | 1.11 | 3 |
| 500:1 | 0.625 | 0.43 | 62.4% | 27200 | 26800 | 1.11 | 2 |
| 1000:1 | 0.539 | 0.32 | 47.7% | 41300 | 49100 | 1.30 | 1.5 |

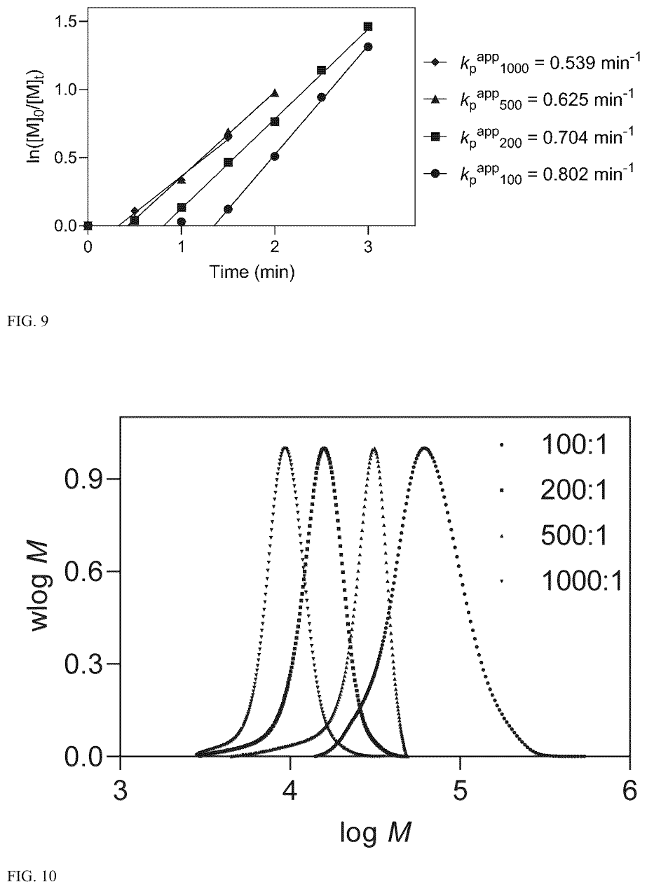

As shown m Table 4, FIG. 9, and FIG. 10, the higher the amount of RAFT agent used, the faster the apparent propagation rate, but the induction period is longer. For both higher monomer ratios (such as 1000:1) and lower monomer ratios (such as 100:1), the RAFT agent demonstrated excellent control. The molecular weight measured by GPC was close to the theoretical value, and the dispersity was good, with D values all below 1.3, indicating that the polymerization falls within the range of controlled polymerization.

### 5. Polymerization of RAFT Agent (RAFT2 Prepared in Example 2) in Different Solvents in Anaerobic Environment

TABLE 5
Compatibility of RAFT Agent with Different Polar Solvents

| | Conversion | M_{n, Theo} | M_{n, GPC} | | Time | |
|---|---|---|---|---|---|---|
| Solvent | rate | (g/mol) | (g/mol) | Ð | (min) | Polarity |
| Acetonitrile (ACN) | 33.3% | 6000 | 6900 | 1.12 | 3 | 6.2 |
| Methanol | 34.5% | 6200 | 7700 | 1.11 | 3 | 6.6 |
| N,N- | | | | | | |
| Dimethylformamide | 55.0% | 9800 | 10000 | 1.16 | 3 | 6.4 |
| (DMF) | | | | | | |
| Tetrahydrofuran | 53.2% | 9400 | 9100 | 1.25 | 3 | 4.2 |
| (THF) | | | | | | |
| Ethyl acetate | 42.5% | 7600 | 9100 | 1.17 | 3 | 4.3 |
| Ethanol | 50.9% | 9100 | 9200 | 1.17 | 3 | 4.3 |
| Dichloromethane | 36.0% | 6500 | 7100 | 1.15 | 3 | 3.4 |
| (DCM) | | | | | | |
| Acetone (Ac) | 31.5% | 5700 | 6500 | 1.13 | 3 | 4.4 |
| Dimethyl sulfoxide | 77.7% | 13700 | 13200 | 1.11 | 3 | 7.2 |
| (DMSO) | | | | | | |

As shown in Table 5, the RAFT agent was able to achieve polymerization in so vents with different polarities, demonstrating that the RAFT agent has good compatibility with a variety of polar solvents. When dimethyl sulfoxide (DMSO) was used as the solvent, the apparent propagation rate was the fastest, with a monomer conversion rate reaching 77.7% after 3 minutes of irradiation.

6. Compatibility of RAFT Agent (RAFT2 Prepared in Example 2) with Different Monomers in Anaerobic Environment

TABLE 6
Compatibility of RAFT Agent with Different
Monomers in Anaerobic Environment

| | Conversion | M_{n, Theo} | M_{n, GPC} | | Time |
|---|---|---|---|---|---|
| Monomer | rate | (g/mol) | (g/mol) | Ð | (min) |
| Methyl acrylate (MA) | 77.7% | 13700 | 13200 | 1.11 | 3 |
| Benzyl acrylate (BzMA) | 87.5% | 28700 | 25200 | 1.09 | 2 |
| N,N′- | 71.2% | 18400 | 19000 | 1.12 | 4 |
| Diethylacrylamide | | | | | |
| (DEA) | | | | | |
| 2-(2-Ethoxyethoxy)ethyl | 69.0% | 26300 | 27800 | 1.33 | 2 |
| Acrylate (DEEGA) | | | | | |
| N-Acryloylmorpholine | 95.2% | 27200 | 22400 | 1.07 | 2 |
| (NAM) | | | | | |
| N,N′- | 65.3% | 13300 | 14100 | 1.1 | 4 |
| Dimethylacrylamide | | | | | |
| (DMA) | | | | | |
| 2-Hydroxyethyl acrylate | 74.4% | 21300 | 30300 | 1.22 | 2 |
| (HEA) | | | | | |
| Butyl acrylate (BA) | 48.4% | 12700 | 12300 | 1.22 | 3 |
| Tert-Butyl | 35.7% | 9500 | 11400 | 1.3 | 2 |
| acrylate (t-BA) | | | | | |
| N-Isopropylacrylamide | 40.0% | 9400 | 11200 | 1.24 | 6 |
| (NIPAm) | | | | | |
| Tetrahydrofuran | 57.0% | 18100 | 20100 | 1.16 | 2 |
| acrylate (TMA) | | | | | |

The structures of the above monomers are as follows:

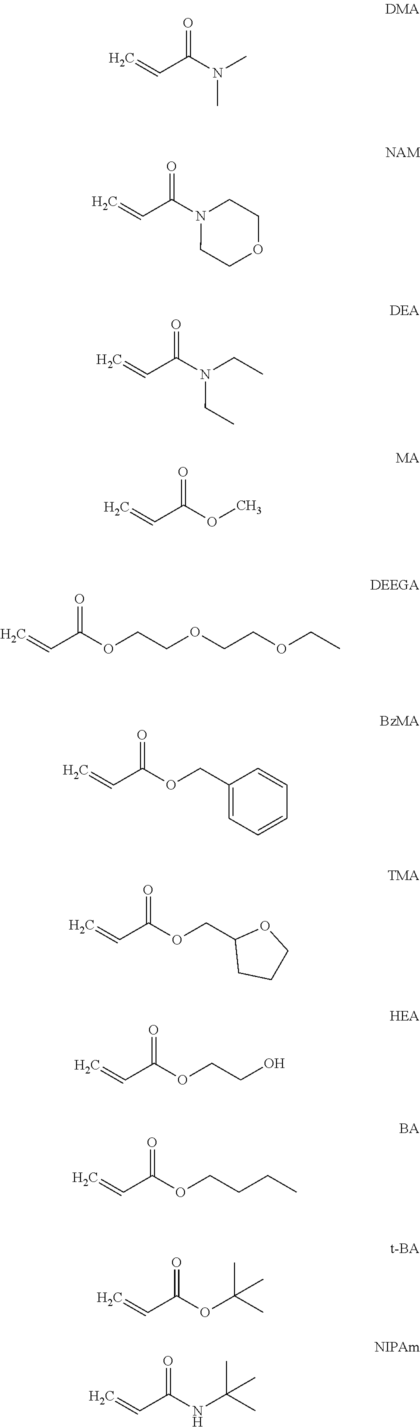

As shown in Table 6, the apparent propagation rates of acrylic acid or acrylate monomers are fast, while the apparent propagation rates of acrylamide monomers are slower. However, for both acrylic acid or acrylate monomers and acrylamide monomers, the apparent propagation rates are significantly higher than those of conventional photo-controlled RAFT polymerization.

### Comparative Example 1

1.1 Preparation of Kinetic Solution: A solution was prepared with a molar ratio of [monomer]:[RAFT agent]=200:1 by mixing the monomer and the RAFT agent, with dimethyl sulfoxide (DMSO) as the solvent. The volume ratio of monomer to solvent was 1:1. The monomer used was methyl acrylate (MA), and the RAFT agent was CDTPA, the structure of CDTPA is as follows:

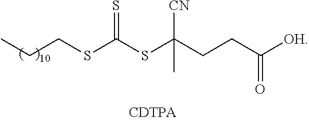

1.2 Kinetic Experiment Procedure: 0.4 mL of the kinetic solution was transferred into a cuvette, sealed with a rubber stopper and sealing film. In anaerobic conditions, the solution was irradiated under a 10 mW/cm² LED light. Fourier Transform Infrared Spectroscopy (FTIR) was used to integrate the vibration absorption peak of the vinyl C—H bond of the monomer near 6200 cm⁻¹. The peak area was used for timed quantitative monitoring of the remaining monomer, thereby tracking the monomer conversion rate. The apparent propagation rate (kₚ^{app}) was calculated using a kinetic equation. Samples were taken when the monomer conversion rates were approximately 40%, 50%, 60%, and 70%, respectively. These samples with different conversion rates were analyzed by Gel Permeation Chromatography (GPC) to check whether the molecular weight during the polymerization process was controllable and to monitor the dispersity of the polymer. After the monomer conversion rate reaches approximately 70% (after the fourth sampling), the polymer was purified. The detection results are shown in Table 7.

### Comparative Example 2

The difference from Comparative Example 1 was that the RAFT agent was DTPA, and ZnTPP was added as a photocatalyst, wherein the molar ratio of [monomer]:[RAFT agent]:[ZnTPP]=200:1:0.01. The structural formulas of DTPA and ZnTPP are as follows:

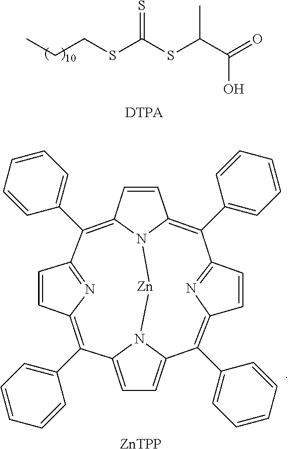

The experimental results are shown in Table 7.

TABLE 7
Polymerization Performances of Comparative Photo-Controlled Systems in Anaerobic Conditions

| | | kₚ^{app} | Induction | | M_{n, Theo} | M_{n, GPC} | | Time | Wavelength |
|---|---|---|---|---|---|---|---|---|---|
| RAFT | PC | (min⁻¹) | Period (min) | Conversion | (g/mol) | (g/mol) | Ð | (min) | (nm) |
| CDTPA | None | 0.008 ± 0.0005 | 29.83 ± 2.81 | 61.6% | 11000 | 10800 | 1.06 | 160 | 460 |
| DTPA | ZnTPP | 0.019 ± 0.0014 | 13.47 ± 2.51 | 64.3% | 11400 | 11100 | 1.05 | 70 | 570 |

As shown in Tables 3 and 7, the polymerization of the reported photo-controlled RAFT polymerization without catalyst, exemplified by CDTPA (Comparative Example 1), had an apparent propagation rate of only 0.008 min⁻¹. In contrast, the RAFT agent of the present invention, without catalyst, only the RAFT agent was replaced, achieved an apparent propagation rate of 0.704 min under the same conditions, which is more than 80 times the apparent propagation rate of CDTPA. Compared to PET-RAFT polymerization, exemplified by DTPA/ZnTPP (0.019 min 1, Comparative Example 2), the apparent propagation rate was more than 35 times higher. The polymerization time was reduced from several hundred or dozens of minutes to less than ten minutes.

The above description is only the preferred embodiments of the present invention and is not intended to limit the present invention. Those skilled in the art can make various modifications and changes to the present invention. Any modifications, equivalent replacements, improvements, etc. made within the spirit and principles of the present invention should be included within the protection scope of the present invention.

1. A reversible addition-fragmentation chain transfer (RAFT) agent for photo-controlled polymerization, having a structure represented by the general formula: 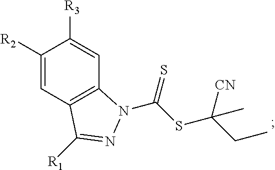
wherein, R₁, R₂, and R₃ are each hydrogen;
or R₂ and R₃ are each hydrogen, and R₁ is methyl;
or R₁ is methyl, R₂ is bromine, and R₃ is hydrogen;
or R₁ is methyl, R₂ is hydrogen, and R₃ is methoxy.

2. The RAFT agent for photo-controlled polymerization according to claim 1, wherein R₂ and R₃ are each hydrogen, and R₁ is methyl.

3. A method for preparing a RAFT agent for photo-controlled polymerization according to claim 1, comprising:
dissolving 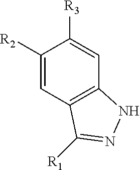
and a metal hydroxide in a first solvent to form a reaction mixture, and keeping the reaction mixture in an ice water bath, then adding carbon disulfide dropwise to the reaction mixture and continuously stirring the reaction mixture in the ice water bath to obtain an intermediate product I;
dissolving the intermediate product I in a second solvent, adding an oxidizing agent, and stirring to obtain an intermediate product II;
dissolving the intermediate product II and 2,2′-Azobisisovaleronitrile (AIBN) in a third solvent, heating, stirring, and introducing nitrogen gas; after the reaction is complete, obtaining the RAFT agent for photo-controlled polymerization;
wherein a structure of the intermediate product I is shown as formula I, and a structure of the intermediate product II is shown as formula II; 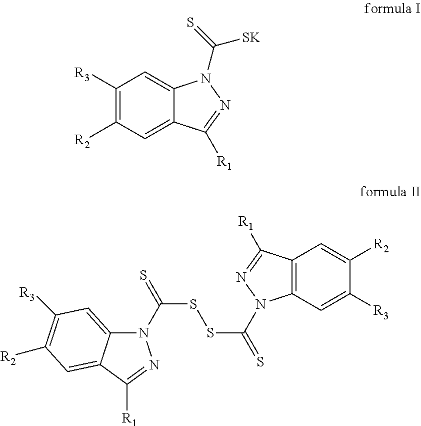
wherein, R₁, R₂, and R₃ are each hydrogen;
or R₂ and R₃ are each hydrogen, and R₁ is methyl;
or R₁ is methyl, R₂ is bromine, and R₃ is hydrogen;
or R₁ is methyl, R₂ is hydrogen, and R₃ is methoxy.

4. The method according to claim 3, wherein the metal hydroxide is selected from at least one of potassium hydroxide and sodium hydroxide;
or the first solvent, the second solvent and the third solvent are respectively selected from tetrahydrofuran, ethyl acetate and acetone;
or the first solvent and the second solvent are tetrahydrofuran, and the third solvent is ethyl acetate.

5. The method according to claim 3, wherein after adding carbon disulfide to the reaction mixture and stirring the reaction mixture, a product is obtained, filtered under reduced pressure, washed with deionized water, and dried to yield the intermediate product I.

6. The method according to claim 3, wherein after adding the oxidizing agent and stirring to complete the reaction, the second solvent and the oxidizing agent are removed, followed by extraction and drying to yield the intermediate product II.

7. The method according to claim 3, wherein the oxidizing agent comprises elemental iodine.

8. The method according to claim 6, wherein a rotary evaporation method is used to remove the second solvent;
or a reducing agent is added to remove the oxidizing agent; preferably, the reducing agent comprises sodium thiosulfate.

9. The method according to claim 3, wherein after heating, stirring, and introducing nitrogen gas until the reaction is complete, the third solvent is removed, and a product is obtained, followed by purification via column chromatography to yield the RAFT agent for photo-controlled polymerization.

10. The method according to claim 9, wherein during the purification via column chromatography, a mixture of petroleum ether and ethyl acetate in a volume ratio of 10:1 is used as a mobile phase.

11. A method for photo-controlled RAFT polymerization, comprising:
mixing a monomer, a RAFT agent for photo-controlled polymerization according to claim 1, and a solvent;
performing a photo-controlled RAFT polymerization reaction under blue light irradiation at 420-430 nm.
